(19) United States
(12) Patent Application Publication
(10) Pub. No.: US 2023/0329721 A1
(43) Pub. Date: Oct. 19, 2023

(54) DEVICE FOR ACHIEVING HEMOSTASIS

(71) Applicant: Cyndrx, LLC, Brentwood, TN (US)

(72) Inventors: John I. Shipp, Jacksonville, FL (US); Denny Lawson, Republic, MO (US)

(21) Appl. No.: 18/044,286

(22) PCT Filed: Oct. 6, 2020
(86) PCT No.: PCT/US20/54435

§ 371 Date: Mar. 7, 2023

Related U.S. Application Data
(60) Provisional application No. 63/078,236, filed on Sep. 14, 2020.

(51) Int. Cl. A61B 17/12

(52) U.S. Cl. CPC A61B 17/12136; A61B 17/1204; A61B 17/12109; A61B 2017/12004; A61M 29/02

(57) ABSTRACT

A percutaneous device for achieving hemostasis in a vessel, particularly a device for achieving hemostasis following the failure of a vascular closure device has a catheter with an attached inflation device. The catheter preferably has two different lumens, one for a guide wire and one for fluid used to inflate the inflation device. When inflated, the inflation device unfurls to block the puncture in the blood vessel. Distal blood flow continues uninterrupted with the device in the furled and unfurled state, preventing any issues distally from the puncture site.

### FIELD OF THE INVENTION

The present invention relates generally to a percutaneous device for achieving hemostasis in a vessel, particularly a device for achieving hemostasis following the failure of a vascular closure device to achieve the desired result. The invention is adaptable to achieve hemostasis following vessel dissection or rupture during cardiac or peripheral catheter procedures.

### TECHNICAL BACKGROUND

For many diagnostic and interventional procedures it is necessary to access arteries or veins. Vessel access is accomplished either by direct vision or percutaneously. In either case, the target vessel is punctured with a hollow needle containing an access guide wire. When the intravascular positioning of the access guide wire has been verified, the hollow needle is removed leaving the access guide wire. Next, a sheath containing a dilator is directed over the access guide wire. The dilator enlarges the puncture opening to facilitate the insertion of the larger diameter sheath into the blood vessel. The sheath usually consists of a hollow tube with an open distal end and a hemostatic valve at a proximal end, which remains outside the body and blood vessel. The hemostatic valve is made of a compliant material and is designed in such a way as to allow devices such as catheters to be inserted and withdrawn from the vessel with minimal blood loss. After the sheath has been inserted into the blood vessel, the dilator is removed leaving a clear passageway in the sheath for the catheter. The sheath is removed from the blood vessel after the procedure is finished resulting in bleeding at the puncture site that must be medically managed.

For small bore (SB) sheaths (4-7 French) traditionally, pressure is applied to the puncture site to allow time for the blood to clot, thereby stopping the bleeding. Depending on the amount of anticoagulants that may have been administered to the patient during and prior to the procedure, the time pressure must be maintained varies from 15 minutes to more than an hour. Once bleeding has stopped, a pressure bandage is placed over the site of the puncture in an attempt to protect the integrity of the platelet-formed clot. The pressure bandage must remain in place for some time, usually from 8 to 24 hours. During this period of time the patient must remain flat in bed, sometimes requiring an overnight hospital stay.

To shorten the length of time required for the patient to become ambulatory and to lessen complications sometimes arising from the traditional method, several vascular closure devices (VCDs) have been developed. One such device is described in U.S. Pat. No. 9,131,931.

A number of percutaneous procedures have been developed that require larger bore openings in the vessels such as valve replacements and ventricular assist devices. These procedures, called structural heart procedures, often require large bore (LB) access sheaths in the range of 12-24 F. The LB device is usually inserted into the femoral artery of either the left or right groin. Usually SB access is gained in the opposite groin for a contrast catheter.

The closure device described in U.S. Pat. No. 9,131,931 is scalable to adapt to the requirements of these structural heart procedures.

In procedures where a SB vascular closure procedure fails, for one reason or another, hemostasis is achieved by reverting to manual compression (MC), the gold standard for hemostasis prior to the advent of VCDs. While hemostasis times are longer with MC, bleeding is stopped in a reasonable period of time. With device failure rates from 1-5% typically and MC available for backup, the failure rates have proven to be acceptable since there is little if any added patient risk.

MC is often not possible for LB device failures owing to the size of the puncture. The risk of a patient bleeding out is simply too high without a reliable backup in cases of LB VCD failures. In case of failure, the wire that is used for access has likely been removed so that there is no longer direct access to the arteriotomy. Additionally, there could be broken or mal-deployed seal components in the tissue track. Further attempts to seal the puncture using the same pathway could result in embolizing these components, making matters even worse.

LB procedures and SB cardiac and peripheral procedures can result in vessel injury such as dissected or ruptured vessels, especially for heavily calcified or torturous vessels. These adverse events require quick hemostasis and repair either from compression clotting, surgical intervention or other means.

For damaged vessels and for malfunctioning LB closure devices the traditional triage for obtaining hemostasis is manual compression (MC), an intra vessel inflatable balloon (angioplasty balloon catheter) which is held in place for temporary hemostasis until clotting is adequate, deployment of a permanent covered stent over the affected area, and surgical intervention. MC is highly effective for hemostasis at the SB arteriotomy site but often ineffective for LB closure device failures and also at SB and LB vessel damage sites. Balloon hemostasis is temporary and has the disadvantage of stopping distal vascular blood flow until the dissection or rupture can be surgically repaired.

What is needed in a case of an LB VCD failure or a SB adverse event is a hemostasis device that can be quickly deployed to achieve hemostasis, is adaptable for a range of vessel diameter, allows blood to continue to flow in the vessel during the hemostasis period, and removable after bleeding has stopped by clotting or surgical intervention.

### SUMMARY OF THE INVENTION

Disclosed herein is a device for obtaining hemostasis of blood exiting a puncture in the wall of a blood vessel, the blood vessel having an interior wall surface, and exterior wall surface, and a lumen. In one aspect, the present invention is directed to a hemostasis device that includes a catheter having a proximal portion, a middle portion, and a distal portion, the distal portion having a distal end, the catheter having a first lumen and a second lumen, the first lumen being sealed at the distal end of the catheter, at least one opening in an outer wall of the catheter between the middle portion and the distal end and into the second lumen, and an inflatable element attached to and in fluid communication with the second lumen of the catheter through the at least one opening, the inflatable element expanding in a radial direction upon insertion of a fluid into the first lumen of the catheter and at least one opening.

The two sides can be affixed in the connected pattern by bonding, heat staking, laser welding, or other known techniques.

In some embodiments, the catheter has an outside wall forming the first lumen and an inside wall within the first lumen and forming the second lumen inside the second lumen.

In some embodiments, the inflatable element is attached along only a portion of a length of the catheter.

In some embodiments, the inflatable element includes at least one channel that is in fluid communication with the at least one opening in the catheter.

In some embodiments, the inflatable element expands in a radial direction upon introduction of a fluid into the first lumen, the inflatable element expanding to contact an internal surface of a blood vessel into which it is inserted.

According to another aspect of the current invention, there is a method of sealing an opening in the wall of a blood vessel, the blood vessel having an interior wall surface, exterior wall surface, and a vessel lumen including the steps of providing a hemostasis device that includes a catheter having a proximal portion, a middle portion, and a distal portion, the distal portion having a distal end, the catheter having a first lumen and a second lumen, at least one opening in an outer wall of the catheter between the middle portion and the distal end and into the second lumen, and an inflatable element attached to and in fluid communication with the second lumen of the catheter through the at least one opening, unserting at least a portion of the hemostasis device into an insertion blood vessel away from the opening in the blood vessel and moving the inflatable element to the location of the opening in the blood vessel, introducing a fluid into the inflatable element through the catheter to expand the inflatable element to block the opening in the wall of the blood vessel.

According to another embodiment of the present invention, there is a hemostasis device for sealing an opening in the wall of a blood vessel, the blood vessel having an interior wall surface, exterior wall surface, and a lumen, the hemostasis device includes a catheter having a proximal portion, a middle portion, and a distal portion, the distal portion having a distal end, the catheter having a first catheter lumen and a second catheter lumen, the first catheter lumen being sealed at the distal portion of the catheter, at least one opening in the catheter between the middle portion and the distal end and in fluid communication with the first catheter lumen, and an inflatable element attached to and wrapped around the catheter, the inflatable element being in fluid communication with the first catheter lumen through the at least one opening, the inflatable element expanding radially upon insertion of a fluid into the first catheter lumen and at least one opening causing an outer wall of the inflatable element to contact the interior wall surface of the blood vessel to at least partially seal an opening in the blood vessel while simultaneously allowing blood to flow past the hemostasis device.

The inflation device used to inflate and deflate (for removal) the inflatable element of the current invention is anyone of a number of such device commercially available such as the BIG60 from Merit Medical.

### BRIEF DESCRIPTION OF THE DRAWINGS

FIG. 1 is a perspective view of a proximal end of one embodiment of a hemostasis device according to the current invention;

FIG. 2 is an elevational view from the proximal end of the hemostasis device of FIG. 1 in the furled state;

FIG. 2A is a cross section of a proximal end of the hemostasis device in FIG. 1;

FIG. 3 is a plan view of the distal end of the hemostasis device of FIG. 1;

FIG. 4 is a cross section of the distal end of an alternative catheter according to another embodiment of the present invention

FIG. 5 is a top perspective view of the distal end of the catheter of FIG. 1 before attachment of the inflation element;

FIG. 6 is a plan view of the inflation element before sealing and installation on the catheter;

FIG. 7 is a view of the inflation element attached to the catheter with 8 sealed channels for inflation fluid; and

FIG. 8 is end view from the proximal end in a blood vessel with the inflation element unfurled and blocking an opening in the blood vessel.

### DETAILED DESCRIPTION OF THE PREFERRED EMBODIMENTS

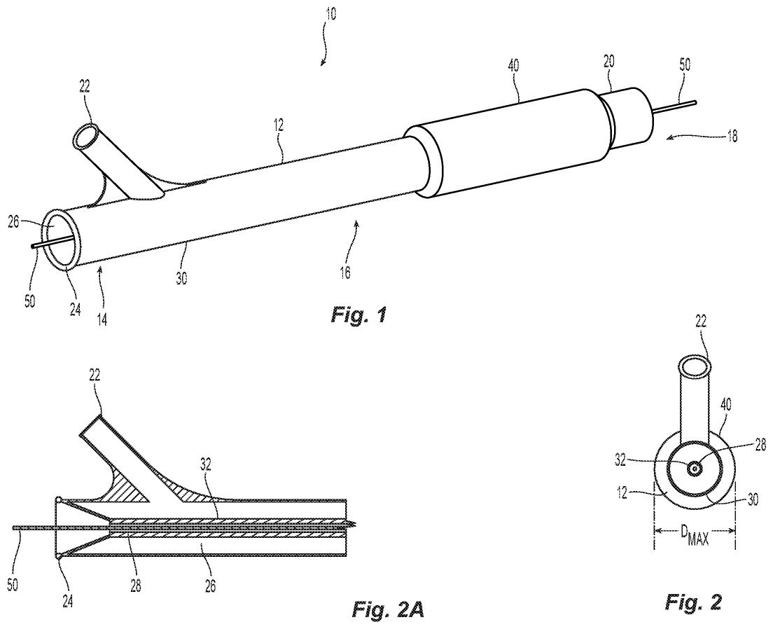

Illustrated in FIG. 1 is a first embodiment of a hemostasis device 10 according to the present invention. The hemostasis device 10 is preferably a catheter 12 that has a proximal portion 14, a middle portion 16 and a distal portion 18, the distal portion 18 having a distal end 20. The catheter 12 may be of any appropriate length for the situation that the device 10 will be used in, as discussed in more detail below. The proximal portion 14 of the hemostasis device 10 includes two ports 22,24 that preferably include Luer Lock connections, although any type of appropriate connectors could be used with the two ports 22,24. The hemostasis device 10 preferably comprises at least two lumens or channels—a first catheter lumen or channel 26 and a second catheter lumen or channel 28, hereinafter first lumen or channel and second lumen or channel. The first lumen 26 is preferably sealed at the distal end thereof. Each of the first lumen or channel 26 and the second lumen or channel 29 are associated with one of the ports 22,24, respectively. The lumens 26,28 may run the length of the catheter 12, but the first lumen 26 may stop short thereof. As illustrated, the second lumen 28 can be centered within the first lumen 26. However, other configurations are also possible as illustrated in FIG. 4 and discussed below. Lumen 28 is a through-channel that begins at the proximal portion 14 of the catheter 12 and extends the length of the catheter 12 to and through the distal end 20. Second lumen 28 is in fluid communication with the port 24. First lumen 26, surrounding second lumen 28, is in fluid communication with port 22, but does not communicate with second lumen 28. Port 22 is used for the introduction of inflation fluid to inflate an inflation element 40 through first lumen 26, as discussed in more detail below.

The catheter 12 has an outer wall 30 that forms the first lumen 26 and an inside wall 32 that forms the second lumen 28 within the first lumen 26. There may be extensions 34 that are used to connect the outside wall 30 to the inside wall 32 (thus being in the first lumen 26) to keep second lumen 28 centered and allow fluid to flow within the first lumen 26 and around the second lumen 28. The second lumen 28 accepts a guide wire 50 through port 24, the guide wire 50 preferably extending beyond the distal end 20 and at least to the opening in the vessel.

An alternative design of a catheter 12′ according to the present invention is illustrated in FIG. 4. Rather than having concentric lumens, the lumens in this catheter are side by side and separated by a septum. The catheter 12′ has a first lumen 26′ and a second lumen 28′. The catheter 12′ also has an outer wall 30′ to form the two lumens. The first lumen 26′ is separated from the second lumen 28′ by a septum 32′. The first lumen 26′ is sealed at the distal end thereof. A guide wire (not illustrated but can be the same as the one illustrated above) is disposed in the second lumen 28′ and extends beyond the distal end of the catheter 12′. Other configurations of the catheter are also possible.

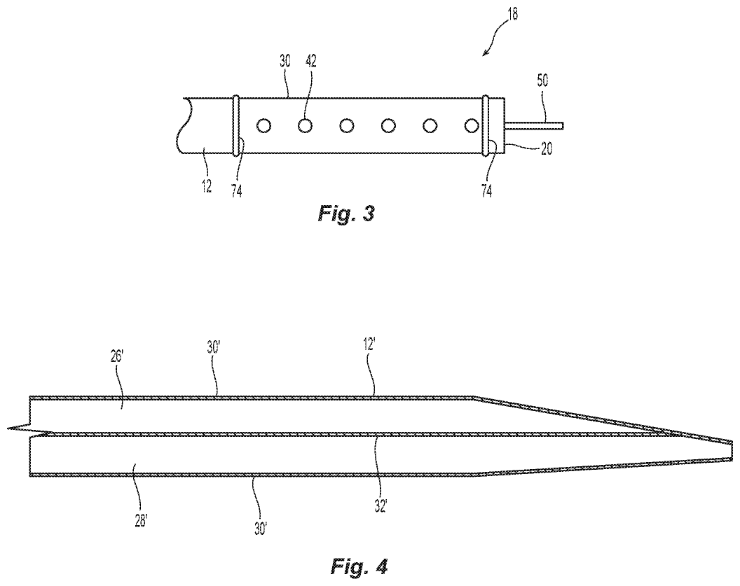

The catheter 12 has at least one opening 42 in the outer wall 30 to allow for the fluid in the first lumen 26 to be routed to the inflation element 40. Preferably, there are a number of openings 42 in the outer wall, six are illustrated in FIG. 3, but there could be more or fewer openings 42 and still fall within the scope of the present invention. It is also contemplated that there could be an elongated opening extending along a length of the catheter 12.

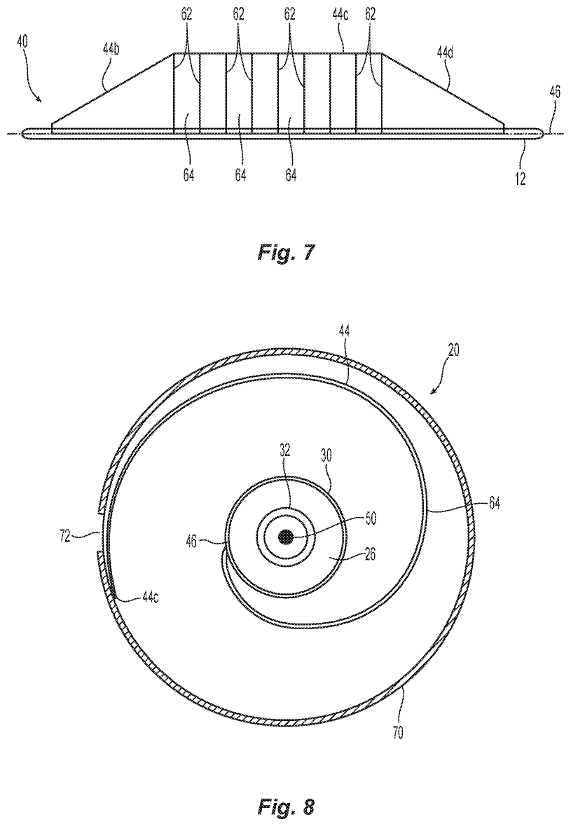

The inflation device 40 can be made from a sheet 44 of polyester or nylon, but the sheet 44 could also be made of other materials, including Pebax, polyurethane, and silicone. Turning to FIG. 6, the sheet 44 has a center line 46 that corresponds to the where the catheter 12 will be located along the sheet 44. While a particular shape of the sheet 44 is illustrated in FIG. 6, any appropriate shape may be used, as long as it performs the functions discussed herein. After the catheter 12 is disposed along the center line 46, the sheet 44 is sealed along its edges 44a,44b,44c, and 44d. As illustrated in FIG. 7, there are also other seals 62 that extend from the catheter 12 outward to the edge 44c. These seals 62 create the channels 64 in the inflation device 40 that allow the inflation device 40 to expand radially outward as the channels 64 are in fluid communication with the first lumen 26. Preferably the largest outside diameter, D_{MAX}, (see also FIG. 2) of the hemostasis device 10, in the furled state is small enough to be accepted in a 6 French or smaller access sheath, about 2 mm or less.

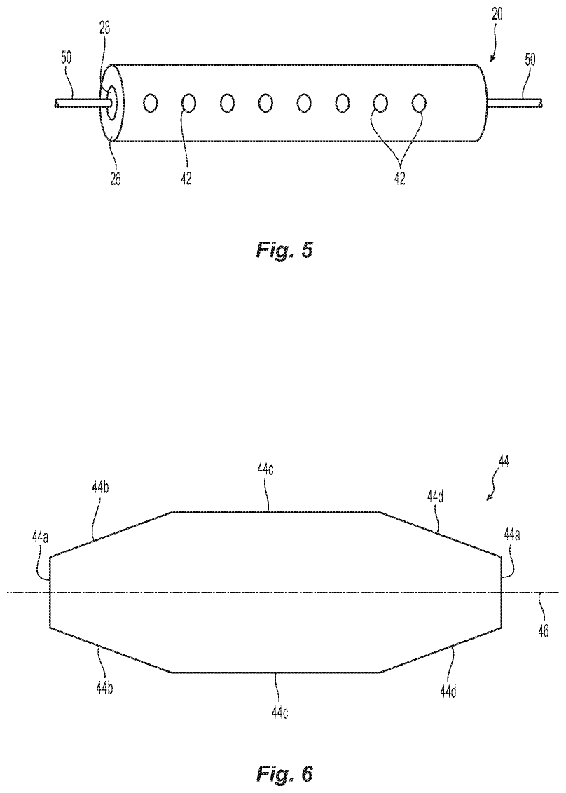

As illustrated in FIG. 5, there is a hemostasis device 10 that has been inserted into a vessel 70 of a patient with a vessel puncture 72. The hemostasis device 10 has been inserted from a different location in the patient's body and delivered by conventional techniques to the location of the vessel puncture 72. Once positioned adjacent the vessel puncture 72, fluid can be introduced to the first lumen 26, which then passes through the opening(s) 42 and into the channels 64 to cause the hemostasis device 10 to radially expand and engage the vessel 70 at the vessel puncture 72. The hemostasis device 10 can remain in position as long as necessary because the blood in the vessel 70 can flow beyond the vessel puncture 72 through the radial expansions of the hemostasis device 10. Once sealed, the hemostasis device 10 can have the fluid removed from the channels 64 and the hemostasis device 10 can be removed from the vessel 70.

It should be noted that the port 24 is for the introduction of the guide wire 50. The device used to inflate and deflate (for removal) the inflation element 40 of the hemostasis device 10 can be any of a number of commercially available devices, such as the BIG60 from Merit Medical. Typically, the fluid used to inflate the device is heparinized saline, but could be inflated with other appropriate fluids as well, such as heparinized saline and contrast mix. The device would be connected to the hemostasis device 10 at port 24, via a standard Luer Lock fitting. Contrast fluid for making the location of the distal end of catheter 12 more visible on a fluoroscope upon insertion into a vessel, can be introduced into either ports 22 or 24 by attaching a standard header used for such tasks (not shown) to either Luer Locks.

There may be other ways in which to locate the various portions of the hemostasis device 10. For example, radio-opaque markers 74 serve to guide, via fluoroscopy, the distal end of catheter 12 such that inflation device 40 is located in juxtaposition with puncture 72 for the purpose of applying pressure for hemostasis at the puncture 72.

The method of use of the invention as a back up device for obtaining hemostasis in case of failure of a LB closure device in a structured heart procedure, for example, is as follows: The structured heart procedure, aorta valve replacement for example, is accomplished by positing and deploying the replacement valve via a 14 F or 18 F catheter from the left or right groin. A 6 F catheter placed in the right or left groin is used to insert contrast fluid aiding visualization under fluoroscopy. After successful replacement the valve catheter is removed from its access sheath. The contrast catheter with the guide wire in place is repositioned to the valve access sight by passing through the femoral artery to the corresponding iliac, entering the valve side iliac, and femoral artery and stationing it upstream of the valve side arrteriotomy. The LB VCD is then deployed and a SB VCD or MC is used to close the contrast side arrteriotomy provided there is no failure of the LB closure. If the LB VCD fails to provide adequate hemostasis catheter 12 of the current invention is rapidly delivered contralaterally via guide wire near the valve side arrteriotomy and inflation device 40 is centered longitudinally on the puncture 72 by means of the radio-opaque markers 74 and contrast fluid, if needed, from a header connected to port 22 or 24. Inflation fluid is injected through first lumen 22 and is distributed through openings 42 and begins to fill channels 62 which causes inflatable element 40 to unfurl. The outer surface of inflation device 40 stops unfurling when the vessel wall is contacted. Inflation fluid flow is terminated when adequate pressure against the puncture site 72 results in hemostasis. Blood continues to flow between the folds to lower extremities. The number of folds in the inflation device 40 is such that full circumference coverage of the largest expected femoral artery diameter, about 12 mm. Hemostasis is achieved in femoral arteries smaller than 12 mm by fewer folds of inflation device 40 unfurling.

In cases of rupture or dissection of cardiac or peripheral procedures the current invention can be used to provide emergency hemostasis by removing the procedure catheter, leaving the guide wire and inserting hemostasis device 10 over the wire. Positioning and unfurling the inflation device 40 is accomplished as described above. As above described, following hemostasis blood is allowed to flow around the furls to the body extremities while the damaged vessel is repaired by surgery or other means.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

What is claimed is:

1. A hemostasis device comprising:
a catheter having a proximal portion, a middle portion, and a distal portion, the distal portion having a distal end;
the catheter having a first lumen and a second lumen, the first lumen being sealed at the distal end of the catheter;
at least one opening in an outer wall of the catheter between the middle portion and the distal end and into the second lumen; and
an inflatable element attached to and in fluid communication with the second lumen of the catheter through the at least one opening, the inflatable element expanding in a radial direction upon insertion of a fluid into the first lumen of the catheter and the at least one opening.

2. The hemostasis device according to claim 1, wherein the catheter has an outside wall forming the first lumen and an inside wall within the first lumen and forming the second lumen inside the second lumen.

3. The hemostasis device according to claim 1, wherein the catheter has an outside wall and the first lumen and the second lumen are separated by a septum.

4. The hemostasis device according to claim 1, wherein the inflatable element is attached along only a portion of a length of the catheter.

5. The hemostasis device according to claim 1, wherein the second lumen receives a guide wire to guide the catheter in a blood vessel.

6. The hemostasis device according to claim 1, wherein the inflatable element includes at least one channel that is in fluid communication with the at least one opening in the catheter.

7. The hemostasis device according to claim 1, wherein the inflatable element expands in a radial direction upon introduction of a fluid into the first lumen, the inflatable element expanding to contact an internal surface of a blood vessel into which it is inserted.

8. The hemostasis device according to claim 6, wherein the at least one opening includes a plurality of openings and the at least one channel includes a plurality of channels, a respective one of each of the plurality of openings being in fluid communication with a respective one of the plurality of channels.

9. A method of sealing a sealing an opening in the wall of a blood vessel, the blood vessel having an interior wall surface, exterior wall surface, and a vessel lumen comprising the steps of:
providing a hemostasis device that includes a catheter having a proximal portion, a middle portion, and a distal portion, the distal portion having a distal end, the catheter having a first lumen and a second lumen, at least one opening in an outer wall of the catheter between the middle portion and the distal end and into the second lumen, and an inflatable element attached to and in fluid communication with the second lumen of the catheter through the at least one opening;
inserting at least a portion of the hemostasis device into an insertion blood vessel away from the opening in the blood vessel and moving the inflatable element to the location of the opening in the blood vessel,
introducing a fluid into the inflatable element through the catheter to expand the inflatable element to block the opening in the wall of the blood vessel.

10. The method of sealing a sealing an opening in the wall of a blood vessel according to claim 9, wherein the insertion blood vessel is the same as the blood vessel with the opening in the wall thereof.

11. The method of sealing a sealing an opening in the wall of a blood vessel according to claim 9, wherein the insertion blood vessel is a different blood vessel as the blood vessel with the opening in the wall thereof.

12. The method of sealing a sealing an opening in the wall of a blood vessel according to claim 9, wherein the inflatable element expands radially to block the opening in the wall of the blood vessel.

13. The method of sealing a sealing an opening in the wall of a blood vessel according to claim 12, wherein blood passes by the inflatable element to reach a distal portion of the blood vessel.

14. A hemostasis device for sealing an opening in the wall of a blood vessel, the blood vessel having an interior wall surface, exterior wall surface, and a lumen, the hemostasis device comprising:
a catheter having a proximal portion, a middle portion, and a distal portion, the distal portion having a distal end;
the catheter having a first catheter lumen and a second catheter lumen, the first catheter lumen being sealed at the distal portion of the catheter;
at least one opening in the catheter between the middle portion and the distal end and in fluid communication with the first catheter lumen; and
an inflatable element attached to and wrapped around the catheter, the inflatable element being in fluid communication with the first catheter lumen through the at least one opening, the inflatable element expanding radially upon insertion of a fluid into the first catheter lumen and at least one opening causing an outer wall of the inflatable element to contact the interior wall surface of the blood vessel to at least partially seal an opening in the blood vessel while simultaneously allowing blood to flow past the hemostasis device.

15. The hemostasis device according to claim 14, wherein the first lumen is sealed at the distal end of the catheter.

16. The hemostasis device according to claim 14, wherein the inflatable element is attached along only a portion of a length of the catheter.

17. The hemostasis device according to claim 14, wherein the second lumen receives a guide wire to guide the catheter in a blood vessel.

18. The hemostasis device according to claim 14, wherein the inflatable element includes at least one channel that is in fluid communication with the at least one opening in the catheter.

19. The hemostasis device according to claim 18, wherein the at least one opening includes a plurality of openings and the at least one channel includes a plurality of channels, a respective one of each of the plurality of openings being in fluid communication with a respective one of the plurality of channels.
